(19) United States
(12) Patent Application Publication
(10) Pub. No.: US 2025/0210145 A1
(43) Pub. Date: Jun. 26, 2025

(54) METHOD FOR DETERMINING THE RESISTANCE OF A MICROORGANISM TO AN ANTIMICROBIAL AGENT

(71) Applicant: Commissariat à l'Energie Atomique et aux Energies Alternatives, Paris (FR)

(72) Inventors: Stéphan PISSOT, Grenoble cedex 09 (FR); Mathieu DUPOY, Grenoble cedex 09 (FR); Pierre MARCOUX, Grenoble cedex 09 (FR)

(73) Assignee: Commissariat à l'Energie Atomique et aux Energies Alternatives, Paris (FR)

(21) Appl. No.: 19/002,668

(22) Filed: Dec. 26, 2024

(30) Foreign Application Priority Data
Dec. 26, 2023 (FR) FR2315333

(51) Int. Cl. G16B 40/10; C12Q 1/18; G16B 40/20

(52) U.S. Cl. CPC G16B 40/10; C12Q 1/18; G16B 40/20

(57) ABSTRACT

A method for training a characterization algorithm, configured to characterize a metabolism of at least one strain of microorganism, the method including the following steps: a) placing different microorganisms of the strain in different training samples, the training samples including a culture medium to which a metabolic marker has been added respectively at different concentrations, the metabolic marker being configured to be metabolized by each microorganism, at least one sample being such that it includes no metabolic marker, or at a concentration considered negligible; b) acquisition of training spectra of microorganisms from each training sample; c) from the training spectra acquired in each training sample, parameterization of the characterization algorithm, so as to characterize, from each training spectrum, a metabolism of the microorganism.

### DESCRIPTION

### Technical Field of the Invention

The technical field of the invention is the determination of a microorganism's resistance to a treatment, in particular an antibiotic or antifungal treatment.

### Background of the Invention

Antibiotic resistance refers to a bacterium's ability to grow in the presence of a certain concentration of an antibiotic agent. It is a public health problem, as some bacteria have already acquired resistance to certain antibiotics. Today, some bacteria are considered to have acquired resistance to a large number of antibiotics.

The sensitivity of a bacterium to an antibiotic refers to the bacterium's ability to grow in the presence of an antibiotic agent. Various methods exist to determine a bacterium's sensitivity to an antibiotic. For example, one such method involves culturing bacteria subjected to different concentrations of antibiotics. However, this process is time-consuming to implement.

Genomic methods have been developed that target gene sequences considered as markers of antibiotic resistance. However, these are expensive methods, and not yet commonly used in everyday practice.

The publication Sharaha U, “Using infrared spectroscopy and multivariate analysis to detect antibiotics'resistant Escherichia coli Bacteria”, Anal. Chem 2017, 89, 8782-8790 describes a method for detecting antibiotic resistance in bacteria using infrared spectrometry, usually referred to by the acronym (FTIR) Fourier Transformed Infrared Spectroscopy. Infrared spectrometry is a measurement technique based on the transmission of infrared radiation through or across the surface of a biological sample. By detecting the characteristic vibrations of chemical bonds, it enables topography of the chemical functions present in the sample. The sample is illuminated by a light source emitting infrared light over a range of wavelengths generally between (25 and 2.5 micrometers), equivalent to a wavenumber between 4000 and 400 cm⁻¹. When the wavelength matches the molecular absorption or absorption of molecules present in the sample, part of the light is absorbed. This results in the detection of absorption peaks by the photodetector.

In the aforementioned publication, a method for detecting antibiotic resistance is described, based on classifying of FTIR-acquired infrared absorption spectra. Classification is performed by a binary classification operator of the SVM (Support Vector Machine) type. The operator is trained beforehand using bacteria, in this case E. Coli, whose antibiotic resistance has already been tested. Implementing the method requires training based on bacteria whose antibiotic resistance has been previously characterized.

In the publication Zwielly A. “Discrimination between drug-resistant and non-resistant human melanoma cell lines by FTIR spectroscopy”, Analyst, 2009, 134, 595-300, FTIR has been reported, to distinguish between melanoma cells resistant and non-resistant to cisplatin, an active ingredient used in chemotherapy. More specifically, spectral differences between cisplatin-resistant and nonresistant cells have been highlighted.

The inventors propose a different method for assessing, preferably non-destructively, the resistance of a microorganism to an antibiotic or antifungal treatment.

### SUMMARY OF THE INVENTION

A first object of the invention is a training method for training a characterization algorithm, configured to characterize a metabolism of at least one strain of microorganism, the method comprising the following steps:
a) distributing different microorganisms of the said strain in different training samples, the training samples comprising a culture medium into which a metabolic marker has been introduced respectively at different concentrations, the metabolic marker being configured to be metabolized by each microorganism, wherein at least one sample does not contain any metabolic marker, or at a concentration considered as negligible;
b) acquiring training spectra of microorganisms from each training sample;
c) from the training spectra acquired in each training sample, parameterizing the characterization algorithm, so as to characterize, from each training spectrum, a metabolism of the microorganism.

According to one embodiment:
in step a), microorganisms of different strains are arranged in different training samples;
step b) comprises acquiring training spectra of microorganisms in each training sample.

The characterization algorithm can be configured to classify the metabolism in a class chosen from at least:
reduced metabolism; and
a normal metabolism.

In step a), the microorganisms can be placed in identical culture media, with the training samples differing from one another by the concentration of metabolic marker added.

The metabolic marker may be an isotopic marker or a chromogenic marker.

According to one possible embodiment, in step b), the microorganisms of each training sample are interposed between an infrared light source and a photodetector, each training spectrum being an infrared absorption or transmission spectrum.

According to one possible embodiment, in step b), the microorganisms of each training sample are arranged on a support and exposed to a laser beam, each training spectrum being a mass spectrum.

A second object of the invention is a test method for determining resistance of a microorganism to an antibiotic or fungicidal agent, the microorganism being arranged in a test sample comprising the metabolic marker used in step a) of a training method according to the first object of the invention, the test method comprising the steps:
i) bringing the microorganism into contact with the antibiotic or fungicidal agent;
ii) acquisition of a test spectrum of the microorganism, the test spectrum being of the same type as the training spectra acquired in each step b) of the training method;
iii) application of a characterization algorithm, as defined in step c) of the training method, to the spectrum resulting from step ii), in order to characterize the metabolism of the microorganism;
iv) determination of the microorganism's resistance to the antibiotic or fungicide based on the characterization performed in step iii).

By test spectrum of the same type, it is meant a spectrum acquired by the same analysis modality.

According to one possible embodiment,
the microorganism is placed in different test samples, each test sample containing different concentrations of the antibiotic agent;
steps i) to iii) are carried out with each test sample;
step iv) comprises determining a concentration of antibiotic or fungicide agent above which the microorganism is no longer resistant.

According to one possible embodiment,
the training method is carried out with training microorganisms;
the test method is carried out with a microorganism different from the training microorganisms.

A third object of the invention is a device, comprising a spectrometer, configured to acquire a test spectrum of a microorganism, the device comprising a processing unit programmed to implement a method according to the second object of the invention.

The spectrometer can be:
an infrared spectrometer;
or a mass spectrometer;
or a Raman spectrometer.

The invention will be better understood on reading the examples of embodiments presented, in the following description, in connection with the figures listed below.

### BRIEF DESCRIPTION OF THE DRAWINGS

FIG. 1 shows the main components of a device configured to implement the invention.

FIG. 2 shows the main stages of a training method and an analysis method.

FIG. 3 illustrates the different stages of the test method.

FIG. 4A shows an absorption spectrum of a sample

FIG. 4B shows a weighting vector resulting from a linear discriminant analysis.

FIG. 4C shows a characterization of metabolism (y-axis), determined by a characterization algorithm, as a function of antibiotic concentration (x-axis). In FIG. 4C, the metabolism of a bacterium used for training has been characterized.

FIG. 5 shows metabolic states (y-axis), determined by a characterization algorithm, as a function of antibiotic concentration (x-axis). In FIG. 5, the metabolism of a bacterium, that was not used during training, has been characterized.

FIG. 6 shows the spectral bands that can be used to implement the invention.

### DETAILED DESCRIPTION

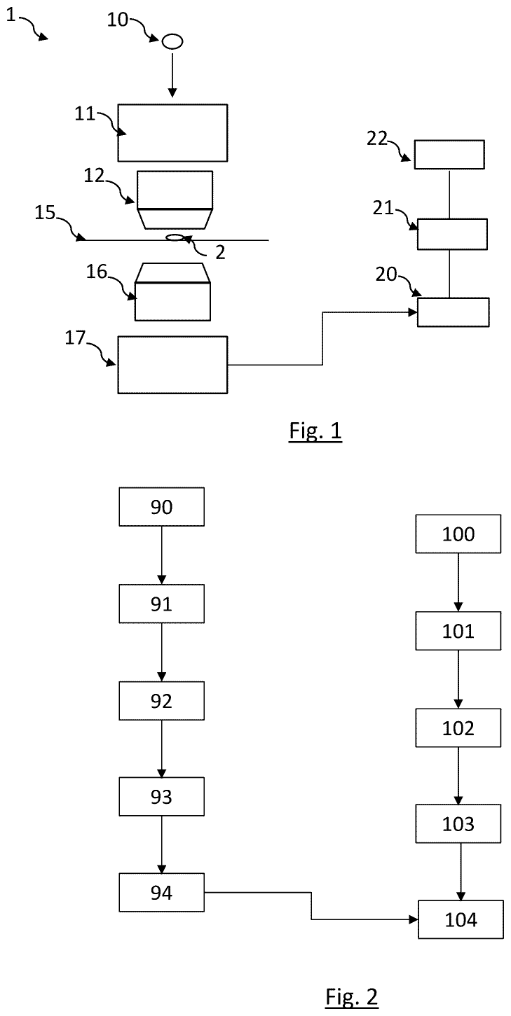

FIG. 1 shows an example of an analysis device 1 for implementing the invention. In this example, the analysis device is an FTIR (Fourier Transform-Infra Red) spectrometer. This type of device, known to those skilled in the art, can be used to acquire a spectrum representative of the transmission of light by a sample. Such a spectrum enables the identification of absorption lines, which constitute a signature of the sample's composition.

The device 1 comprises a light source 10 configured to emit a light beam, referred to as the incident light beam, towards a sample 2. The light beam is emitted in an infrared spectral band, typically between 2.5 μm (i.e. a wave number of 4000 cm⁻¹) and 16 μm (i.e. a wave number of 600 cm⁻¹). The device 1 also comprises an interferometer 11. The beam from the interferometer is focused onto the sample 2 by a first objective 12. The beam transmitted by the sample 2 is collected by a second objective 16, which is optically coupled to a photodetector 17.

The photodetector 17 is connected to a processing unit 20, programmed to produce a spectrum corresponding to the intensity of light transmitted by the sample as a function of wavelength. The spectrum reveals absorption lines, which specifically correspond to chemical bonds within the sample. The processing unit 20 comprises a microprocessor programmed to form the spectrum from the measurements resulting from the photodetector 17. The processing unit 20 is connected to a memory 21, in which the spectrum processing instructions have been stored. The term processing unit is to be understood in a broad sense. It may be one or more microprocessors connected physically to the photodetector 17, or by a wireless link.

In this example, the sample 2 is a drop containing the microorganisms to be analyzed. The drop is deposited on a transparent slide 15 acting as a sample support. The slide 15 is transparent to the incident beam.

The term ‘microorganisms’ refers not only to bacteria but also to fungi, microalgae or prokaryotes. In the following example, the microorganism is a bacterium.

In the prior art, a method has been described, based on a training step, in which bacteria are placed in a culture medium containing an antibiotic. The aim is to determine a spectral signature of the antibiotic's action. This implies building up a database of spectra representative of the antibiotic's degradation of the bacteria. It can be assumed that the spectral signature may vary according to the bacterial strain and the antibiotic used. As a result, databases specific to bacterial strain/antibiotic pairs are required.

The approach taken by the inventors is different. The aim is to obtain a spectral signature that reflects a change in the metabolism of any bacterium, whatever the antibiotic used.

Metabolism refers to all enzymatic reactions taking place within a cell. To survive and multiply, bacteria constantly transform molecules from their environment, producing more complex biomolecules. Consequently, quantifying the effect of an antibiotic agent on these reactions provides information on a bacterium's sensitivity to a given concentration of antibiotic, without having to wait for it to grow.

In order to monitor the evolution of metabolism, the bacteria are placed in a culture medium containing a metabolic marker. The metabolic marker can be an isotopic marker (e.g. deuterium ²H, usually denoted D, or ¹³C), or a molecule that absorbs infrared light at a predetermined wavelength. When using an isotopic marker, D can be incorporated into water (heavy water). ¹³C can be incorporated into glucose. The function of the metabolic marker is to be able to track, by an analysis method, and in particular a spectral analysis method, changes in the bacteria's metabolism. Preferably, the spectral analysis method is non-destructive. Accordingly, the preferred analysis method is FTIR spectrometry. The invention can be applied to other spectral analysis methods, as described below.

Generally speaking, the method is essentially based on the use of a classifier that, from a test spectrum, such as an FTIR spectrum, makes it possible to evaluate the metabolism of a bacterium. More precisely, the classifier is programmed to classify a bacterium, on the basis of the test spectrum, into a class representative of its metabolism. Characterization can enable classification in one class of at least 2 classes, corresponding either to the presence of metabolism or the absence or reduction of metabolism, in which case the bacterium is considered dead or degraded. Preferably, classification is carried out between different classes, each class corresponding to a certain level of metabolism.

FIG. 2 shows the main steps in a training phase, used to parameterize the classifier. The steps are referenced 90, 91, 92, 93 and 94.

Step 90: The bacteria from the same strain, or from different strains, are distributed to form different training samples. Each training sample contains the same culture medium. Some training samples contain the metabolic marker, the concentration of which is known and varies between the different samples. Some training samples contain no metabolic marker, or only a negligible concentration.

Prior to step 90, a pre-culture can be carried out, to synchronize the metabolic states of the bacteria that will be distributed in the different training samples. The pre-culture can last several hours, for example overnight. As the bacteria are placed in the same culture medium for a fairly long period of time, their respective metabolisms can be considered comparable at the end of the preculture: this is known as metabolic synchronization.

Preferably, a wide variety of bacterial strains are used, for example gram-positive, gram-negative, coccus/bacillus strains.

Stage 91: incubation. In this step, each training sample is incubated for a few hours, e.g. two hours.

Stage 92: Washing: the training samples are washed, i.e. centrifuged and resuspended in water. This allows residues of metabolic markers not consumed by the bacteria to be removed from each training sample.

Stage 93: acquisition of training spectra. Training spectra resulting from each washed sample are acquired. In this example, the training spectra are FTIR spectra.

Following step 93, a large number of training spectra are available, from both labeled and unlabeled bacteria. In order to be able to characterize the labeling, it is preferable, although not necessary, for steps 90 to 93 to be carried out using a variety of bacterial strains. In this way, training spectra are obtained which are respectively representative of labeled bacteria, possibly with different levels of labeling, and unlabeled bacteria. The use of a variety of bacterial strains enables better characterization of the labeling by the characterization algorithm described below. The aim is to obtain a characterization that is as universal as possible, and not specific to the metabolism of a particular strain.

Step 94: parameterization of the characterization algorithm. In this step, the characterization algorithm is trained, using the different training spectra resulting from step 93. Each spectrum is associated with a known labeling level: absence of labeling, presence of labeling, and possibly different labeling levels.

The aim of the training is for the characterization algorithm to use a spectrum to characterize a labeling level: absence of labeling or presence of labeling, and possibly different non-zero levels of labeling. In this way, each class is representative of a labeling level, two different classes being respectively representative of two different labeling levels. At least one class is representative of a zero labeling level, or can be considered as such. When using the method on unknown samples, the labeling level can be translated into a metabolism level: the higher the labeling level, the more active the metabolism.

The characterization algorithm can be a classification algorithm, such as Linear Discriminant Analysis (LDA), a neural network, or Partial Least Square (PLS) regression.

The characterization algorithm, once parameterized, is intended to be used to analyze the metabolism of a bacterium in the presence of an antibiotic and the marker used during training, on the basis of a test spectrum. The presence of active metabolism indicates resistance to the antibiotic. The absence of metabolism, reflected by a zero label level, indicates sensitivity to the antibiotic.

In contrast to prior art:
the characterization algorithm is not trained in the presence of an antibiotic;
the characterization algorithm is preferably trained using different bacterial strains. Preferably, the same bacterial strain should be labeled and unlabeled, i.e. placed in training samples with and without metabolic markers.

Once the characterization algorithm has been parameterized, the antibiotic resistance analysis steps can be carried out. This involves testing the metabolism of a bacterium in the presence of an antibiotic, preferably at different antibiotic concentrations. The aim is to determine the concentration at which the bacteria no longer metabolize, or metabolism is reduced.

In a step 100, bacteria of the same strain are placed in different test samples. It is preferable to use bacteria that were used during training, although this condition is not strictly required. Each test sample contains the same culture medium, preferably similar to that used during the training phase. The culture medium contains the same metabolic marker as that used during the training phase. The test samples contain different concentrations of an antibiotic.

In step 101, each test sample is incubated, preferably for the same or a similar time as the incubation time in the training phase. See step 91.

During an incubation step 102, each test sample is collected, then washed and placed on the slide 15.

In an analysis step 103, each test sample is analyzed to acquire a test spectrum. At least as many test spectra are thus obtained as test samples. Preferably, several test spectra, typically several dozen, are acquired for each test sample, and possibly averaged.

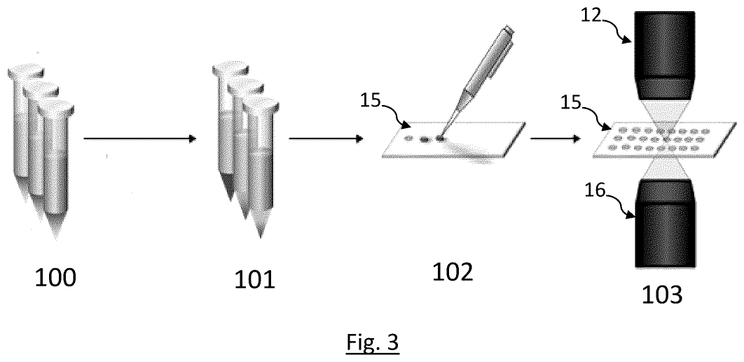

Steps 100 to 103 are illustrated in FIG. 3.

In step 104, the characterization algorithm parameterized during the training phase is applied to each test spectrum, in order to characterize the sample's metabolism. Test spectra corresponding to test samples with a low antibiotic concentration are considered representative of active metabolism: this means that the bacteria are metabolizing the culture medium in the presence of the antibiotic. test spectra corresponding to samples with a high antibiotic concentration are considered representative of slow or inactive metabolism: this means that the bacteria die and do not metabolize the culture medium in the presence of the antibiotic.

The characterization algorithm distinguishes between normal metabolism, which corresponds to no disruption of the bacterium's metabolism, and a slowed metabolism, which corresponds to a slowing down of the bacterium's metabolism under the effect of the added antibiotic concentration. During training, the classes corresponding respectively to normal metabolism and slowed metabolism are set up using training samples in which the metabolic marker concentration is respectively high and low. It is understood that the metabolic marker concentration, i.e. the labeling level, is representative of metabolic activity.

Comparison of the characterizations of each analytical sample enables to determine a concentration, above which the bacteria are considered sensitive to the antibiotic.

The inventors have implemented the training (steps 90 to 94) and test (steps 100 to 104) phases.

Eight strains of different bacterial species were used as training samples (Escherichia. coli, Klebsiella. aerogenes, Citrobacter. freundii, Saphylococcus. epidermedis, Pseudomonas putida and Saphylococcus. lentus).

For each of these strains, an overnight pre-culture was carried out to synchronize the metabolic states of the bacteria, then two two-hour cultures were made from that pre-culture: one in 2 mL of Mueller-Hinton medium (the reference culture medium for performing antibiograms) and 50% v/v heavy water, and one in Mueller-Hinton medium alone, without heavy water.

After two hours' incubation, the training samples were centrifuged and re-suspended in 30 μL of water, then a 5 μL drop was placed on a calcium fluoride (CaF2—infrared-transparent) slide acting as an analysis support. The slide was dried by exposure to a hot and dry atmosphere.

For each training sample, 64 spectra were acquired using an FTIR microscope at 15× magnification. The high number of spectra was chosen to eliminate potential variations in the sample's thickness or texture. For each training sample, a training spectrum was formed by averaging the acquired spectra.

The training spectra obtained were organized into two classes: those corresponding to “labeled” samples (culture medium containing heavy water) and those corresponding to unlabeled samples. A partial least squares (PLS) regression was trained on this dataset. The result is a model capable of quantifying the heavy water labeling of a sample by its FTIR spectrum.

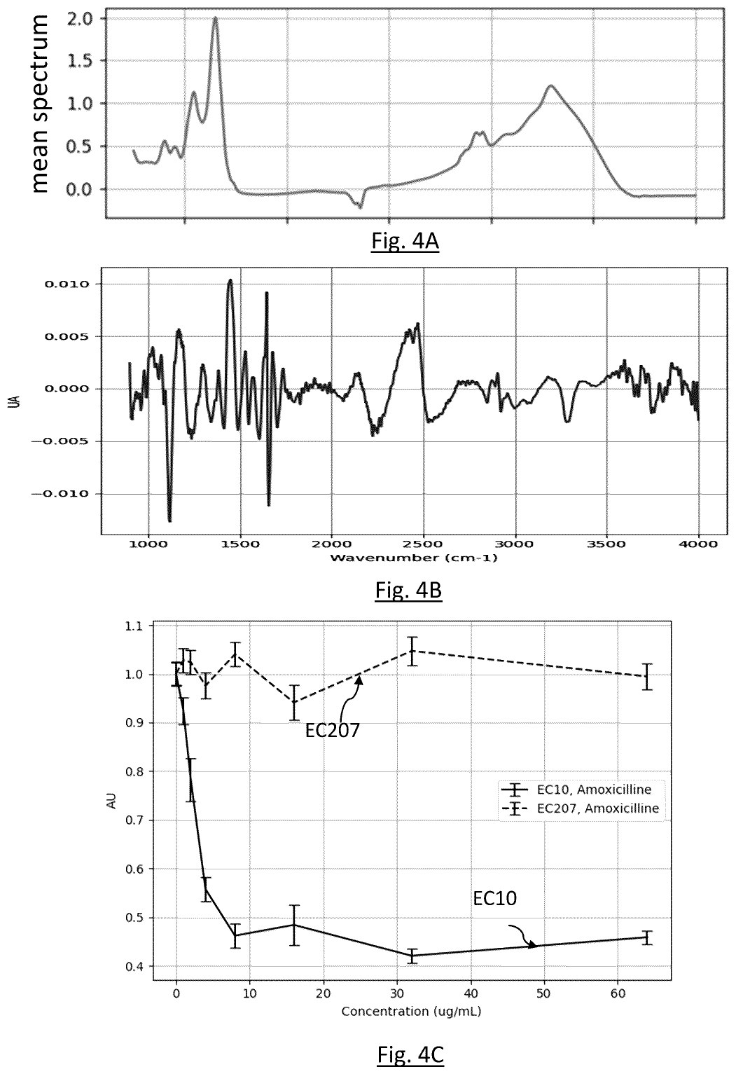

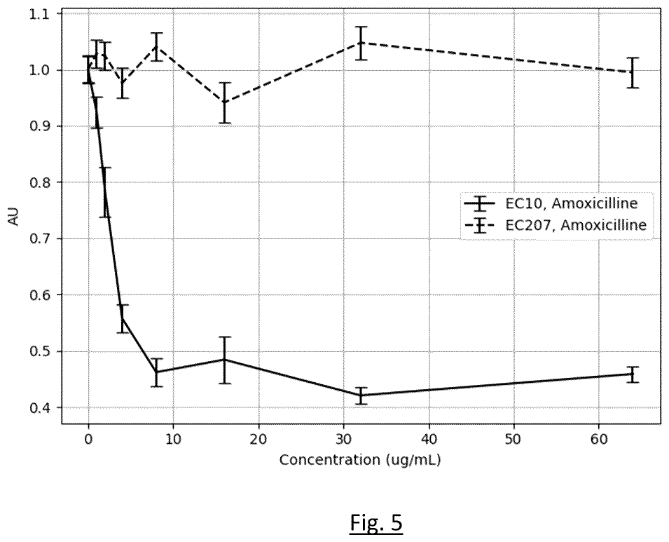

FIG. 4A shows an example of the absorption spectrum of a training sample containing a Deuterium-labeled Mueller-Hinton culture medium. The x-axis corresponds to the wave number (unit cm⁻¹).

FIG. 4B shows an example of a weighting vector produced by linear discriminant analysis (LDA) on training spectra. This vector represents the weights (y-axis) assigned to each wavenumber (x-axis) to separate populations incubated with heavy water from those incubated without heavy water. In the vector shown in FIG. 4B, the wave numbers corresponding to the weights with the highest absolute values are considered the most discriminating between two classes.

Once the LDA classifier had been parameterized, it was used in a test phase on Escherichia coli cultures. Two reference strains were used: strain ATCC25922, referred to as “EC10”, is sensitive to amoxicillin, exhibiting growth inhibition above a concentration of 4 μg/mL of the antibiotic; strain ATCC35218, referred to here as “EC207”, is resistant to amoxicillin.

An overnight pre-culture was performed to synchronize the metabolic states of the bacterial populations. Test samples were prepared using different concentrations of amoxicillin (64, 32, 16, 8, 4, 2 and 1 μg/mL), and a culture of each strain was prepared in 2 ml of Mueller-Hinton medium with 50% v/v heavy water and antibiotic. A control sample without antibiotic was prepared. The test samples were then incubated for two hours. After incubation, the test samples were centrifuged and re-suspended in 30 μL of water, then a 5 μl drop was placed on a calcium fluoride slide. The slide was dried by exposure to a hot, dry atmosphere. On each test sample, 64 spectra are acquired using an FTIR microscope at 15× magnification, in order to eliminate potential variations in the sample's thickness of texture.

This provided test spectra corresponding to the exposure of bacterial strains to different antibiotic concentrations. The algorithm parameterized during the training phase enabled measuring a labeling level for each sample, which is considered representative of metabolic activity. FIG. 4C shows the labeling level for each Amoxicillin concentration. In FIG. 4C, the characterization results for EC10 and EC207 have been identified.

When the labeling level decreased at high antibiotic concentrations the bacterium was considered as sensitive to the antibiotic. Conversely, when the labeling level remained constant, even in the presence of a high antibiotic concentration, the bacterium was considered as resistant. In FIG. 4C, it can be observed that strain EC207 is resistant to all the antibiotic concentrations used, as metabolism is maintained at a high level for all antibiotic concentrations. Strain EC10 is also sensitive to antibiotics above a certain concentration, of the order of 10 μg/mL. In FIG. 4C, the unit of the x-axis is the antibiotic concentration in the sample (μg/mL).

One advantage of the method is that the training phase can be carried out using different bacterial strains, with the same metabolic marker, bearing in mind that it is preferable for the culture medium used in the test phase to match the culture medium used in the training phase. This improves the robustness of the algorithm. It may also enable the algorithm to be applied to samples containing bacterial strains not used in the training phase

FIG. 5 shows an example of characterizing a metabolism by implementing the characterization algorithm as previously described, with two strains of a Staphylococcus Saprophyticus bacterium (SS91, SS96) not used during training. The x-axis corresponds to the antibiotic concentration (Gentamicin). The y-axis corresponds to a labeling level. It can be observed that the algorithm can determine, for each strain, an antibiotic concentration above which the labeling level decreases, reflecting a slowdown in metabolism.

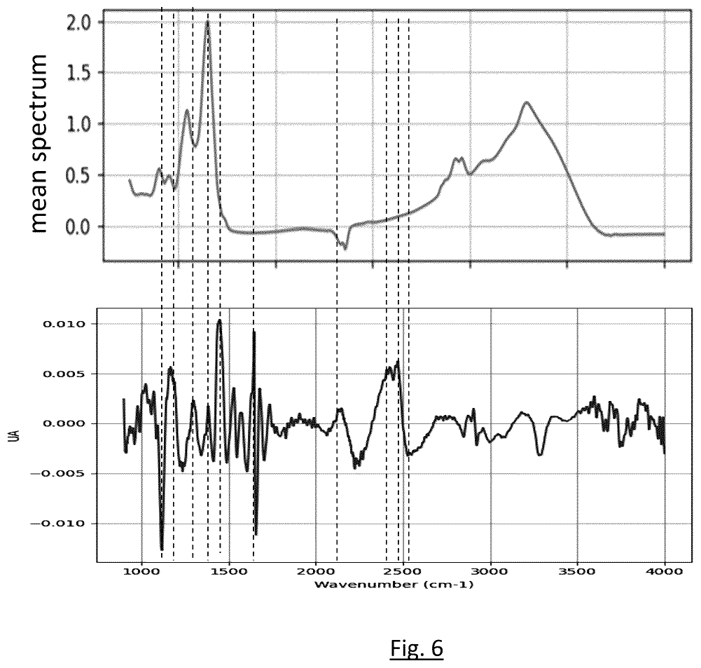

In the above example, training and test spectra extending continuously between 1200 cm⁻¹ and 4000 cm⁻¹ have been described. The invention can be implemented with certain spectral bands only, for example spectral bands of interest, in which the classifier enables better distinction between different classes. In FIG. 6, spectral bands of interest have been represented by dotted lines. The curves shown in FIG. 6 correspond to those described in connection with FIGS. 4A and 4B. The spectrum may be extracted by restricting to the spectral bands of interest.

Although described in relation to the interaction of a bacterium and an antibiotic, the invention can be applied more generally to the analysis of bacterial interactions with bactericidal or bacteriostatic agents.

The invention can also be applied to the analysis of interactions between fungi and a fungicidal agent, or between other types of microorganisms (microalgae, prokaryotes) and a biocidal agent.

Furthermore, although described in relation to the acquisition of spectra using an FTIR modality, the invention may be applied to other spectral analysis methods, in transmission or reflection, whether non-destructive or destructive. This includes: Attenuated Total Reflectance, Raman spectrometry (which can be destructive) or mass spectrometry (destructive), for example in MALDI-TOF mode (Matrix Assisted Laser Desorption Ionization-Time of Flight).

The invention can be implemented by acquiring a spectrum representative of the composition of the microorganisms. The metabolic marker is selected so that it can be identified by the spectrum.

1. A method for training a characterization algorithm, configured to characterize a metabolism of at least one strain of microorganism, the method comprising the following steps:
a) placing different microorganisms of the said strain in different training samples, the training samples comprising a culture medium into which a metabolic marker has been introduced respectively at different concentrations, the metabolic marker being configured to be metabolized by each microorganism, wherein at least one sample does not contain any metabolic marker, or at a concentration considered as negligible;
b) acquiring training spectra of microorganisms from each training sample;
c) from the training spectra acquired in each training sample, parameterizing the characterization algorithm, so as to characterize, from each training spectrum, a metabolism of the microorganism;
wherein in step b) is carried out without antibiotic in each training sample.

2. The method according to claim 1, wherein:
in step a), microorganisms of different strains are arranged in different training samples;
step b) comprises acquiring training spectra of microorganisms in each training sample.

3. The method according to claim 1, wherein the characterization algorithm is configured to classify the metabolism into a class selected from at least:
a reduced metabolism; and
a normal metabolism.

4. The method according to claim 1, wherein in step a) the microorganisms are placed in the same culture medium, the training samples differing from one another by the concentration of metabolic marker added.

5. The method according to claim 1, wherein the metabolic marker is an isotopic marker or a chromogenic marker.

6. The method according to claim 1, wherein in step b), each training sample is placed between an infrared light source and a photodetector, each training spectrum being an infrared absorption spectrum or an infrared transmission spectrum.

7. The method according to claim 1, wherein in step b), each training sample is placed on a support and exposed to a laser beam of a mass spectrometer, each training spectrum being a mass spectrum.

8. A test method for determining resistance of a microorganism to an antibiotic or fungicide agent, the microorganism being placed in a culture medium comprising the metabolic marker used in step a) of the training method according to claim 1, the test method comprising the steps of:
i) bringing the microorganism into contact with the antibiotic or fungicidal agent;
ii) acquiring a test spectrum of the microorganism, the test spectrum being of the same type as the training spectra acquired in each step b) of the training method;
iii) applying the characterization algorithm, as configured in step c) of the training method according to claim 1, to the test spectrum resulting from step ii), thereby characterizing the metabolism of the microorganism;
iv) determining the microorganism's resistance to the antibiotic or fungicide agent based on the characterization performed in step iii).

9. The test method according to claim 8, wherein:
the microorganism is placed in different test samples, each test sample comprising different concentrations of the antibiotic, each test sample comprising a culture medium comprising the metabolic marker used in step a) of the training method;
steps i) to iii) are carried out with each test sample;
step iv) comprises determining a concentration of antibiotic or fungicide agent above which the microorganism is no longer resistant.

10. The test method according to claim 8, wherein
the training method is carried out with training microorganisms;
the test method is carried out with a microorganism different from the training microorganisms.

11. A device, comprising a spectrometer, configured to acquire a test spectrum of a microorganism, the device comprising a processing unit programmed to implement step iii) of the test method according to claim 8.

12. The device according to claim 11, wherein the spectrometer is
an infrared spectrometer;
or a mass spectrometer,
or a Raman spectrometer.
